(19) United States
(12) Patent Application Publication
(10) Pub. No.: US 2007/0116176 A1
(43) Pub. Date: May 24, 2007

(54) X-ray device for making an image of a part to be examined

(76) Inventors: Oliver Meer, Munchen (DE); Martin Ramsauer, Pyrbaum (DE)

Correspondence Address: BRINKS HOFER GILSON & LIONE, P.O. BOX 10395, CHICAGO, IL, 60610, US

(21) Appl. No.: 11/595,106

(22) Filed: Nov. 9, 2006

(30) Foreign Application Priority Data
Nov. 18, 2005 (DE) 10 2005 055 175.0

(51) Int. Cl. A61B 6/04

(52) U.S. Cl. 378/37

(57) ABSTRACT

An X-ray device for making an image of a body part to be examined is provided. The X-ray device includes a support that is operative to support an emitter head having a beam region between an X-ray source, and an X-ray detector. The body part to be examined is positioned in the beam region. A guard device includes a shield and a mount. The mount couples the shield to the support and is coupled to the support at substantially the level of the shield. The guard device is operative to guard against penetration of an unexamined body part into the beam region.

The present patent document claims the benefit of the filing date of DE 10 2005 055 175.0, filed Nov. 18, 2005, which is hereby incorporated by reference.

### BACKGROUND

1. Field

The present embodiments relate to an X-ray device for making an image of a part to be examined.

2. Related Art

The present embodiments relate in general to the improvement of X-ray equipment, for example, the field of medical technology. Generally, during examinations using X-radiation for diagnosis, unexamined parts of a subject are not hindered from moving relatively freely and are potentially radiated with the X-radiation. The conventional X-ray equipment increases the possibility that the subject to be examined will be subjected to an unnecessarily large amount of X-radiation, and the results of the X-ray examination for the part to be examined of the subject are made incorrect. For example, such problems are significant in the field of mammography, in which the subject of the X-ray examination is as a rule the female breast.

The process of mammography is an imaging process that detects lumps, for example, tumors, or hardening of parts or of the entire breast. The examination of diseased changes in breast tissue by mammography is called a diagnostic test. The process of mammography is also employed for palpably enlarged lymph nodes in the armpit or in precautionary examinations for breast cancer, which are known as screening exams. Screening exams are used as a precautionary measure, for example, even when no changes in the breast tissue have been noticed (occurred). For example, screening exams are in contrast to the diagnostic tests for diseased tissue changes that have already occurred. With increasing health awareness, mammography is of growing importance in cancer screening.

Generally, when a mammogram is being made a female patient stands freely in front of a mammography device. During the examination, the patient's breast is fixed in a compression unit of the mammography device. If the patient tilts her head forward during the examination, for example, her head enters into the X-radiation. The X-radiation is located between an X-ray source and an X-ray detector during the examination.

From Published German Patent Disclosure DE 10 2005 015 531 A1, a stationary tomographic mammography device is known which has a detector, an arc-shaped mounting system that has a number of X-ray emitters connected with it. No protective measures are taken for protecting the patient against unnecessary radiation exposure in this system.

### SUMMARY

The present embodiments may obviate one or more of the limitations of the related art. For example, in one embodiment, an X-ray device can lessen the radiation exposure of a subject to be examined.

In one embodiment, an X-ray device includes a guard device that guards against penetration of an unexamined part of the subject into the beam region. The guard device includes a shield and a mount for mounting the shield on the support. The mount is attached to the support substantially at the level of the shield.

In one embodiment, a guard device is provided for one or more body parts of the subject, for example, the head, arms, legs, or genital region. The properties of the guard device can be adapted to the hardness and intensity of the X-radiation employed and to the body parts to be protected. The adaptation of the guard device can be assured, for example, by the shape, material, and production process of the guard device. The guard device reduces the unnecessary radiation dose to the subject, and increases the patient's safety.

In one embodiment, the guard device includes a shield and a mount for mounting the shield on the support. The guard device may be adapted to the given conditions of the X-ray device in question. For example, the mount is shaped and attached depending on the requirements of the X-ray device, such as where it is located and its embodiment. In one exemplary embodiment, the mount is embodied on a wall defining the examination room. The mount is not limited to this location, for example, the mount can be embodied on various components of the X-ray device. The mount and the shield can also be of different materials. For example, the mount and the shield have different materials because they must meet different requirements in terms of load and usage.

In another embodiment, the mount is attached to the support substantially at the level of the shield. In this embodiment, the guard device is compact in size. For example, the mount presents hardly any hindrance, if any, to examining the subject or to the work of the medical staff in conjunction with the examination to be made with the X-ray device. Complicated embodiments of the mount can be dispensed with.

In one embodiment, the guard device is embodied in one piece. In this embodiment, the guard device can be manufactured and fixed compactly, quickly, and simply. For example, the guard device can be manufactured by a casting process. The mount and the shield need not be joined together using an additional connecting piece that is vulnerable to becoming soiled. The cleanliness and sterility of the X-ray device is thus enhanced.

In one embodiment, at least the shield is embodied in transparent form. In this embodiment, neither the patient's vision nor the medical staff's view of the patient is impaired by the guard device. For example, the entire guard device (i.e. the mount and the shield) can be embodied in transparent form and in one piece. The guard device can include any suitable material. For example, Plexiglass or other transparent, castable plastics can be used as the guard device material.

In one embodiment, at least the shield is attached displaceably and/or rotatably to the support. The position in terms of the height and/or inclination of the shield can be adapted to the height and stature of the patient. The shield or the entire guard device can, if needed, be pivoted out of the way because of the rotatable support. In this embodiment, the rotatable support enables free access to the X-ray device or to individual components of the X-ray device.

In one embodiment, the shield is a circular-annular segment. In this embodiment, the shield is embodied to protect the patient's head from the X-radiation. The shield additionally protects against collision of the patient's head with the X-ray source if the X-ray source is disposed rotatably. When the X-ray source is disposed rotatably, the function of the guard device is expanded. In one embodiment, the length of the arc of the circular-annular segment is adapted to the angular range of rotation of the X-ray source.

In one embodiment, the shield has a visor. The visor is a component of the shield. The visor extends, for example, over the entire field of view of the patient. In one embodiment, the visor is a component of the one-piece guard device. The visor can be attached replaceably. In one embodiment, the visor is embodied in transparent form; however, the visor is not limited to a transparent form. The visor further enhances the protection of the patient.

In one embodiment, at least the shield comprises a material that shields against X-rays. In this embodiment, the unexamined body parts of the patient are prevented from getting into the beam region and scattered X-radiation is also prevented from penetrating through the shield into the body of the patient. In one embodiment, the shield includes a lead matrix of suitable concentration. The lead matrix can be built into the shield or into the mount during the production of the shield. In this embodiment, the intensity of the scattered X-radiation is further reduced by the lead present in the shield.

In one embodiment, the mount is attached to the support by a detent connection. In this embodiment, the mount and the guard device are less vulnerable to shock and impact, which increases the safety of the guard device. The guard device is attached in such a way that it can withstand heavier loads. The detent connection enables fast removal of the mount and of the guard device, which enhances the flexibility of the X-ray device. Alternatively, a screw connection or plug-in connection can replace the detent connection. A combination of the detent connection, screw connection and plug-in connection for mounting the mount is also possible. The support is understood to be all the components of the X-ray device that are present for the particular use of the X-ray device and that are not embodied as an X-ray source and an X-ray detector.

In one embodiment, the mount includes a clasp element, which via its support face follows the course of the contour of the support. In this embodiment, the detent, screw or plug-in connections are not required. For example, a suitable distribution of the weight of the shield and the mount makes a stable position of the guard device possible, which protects the patient against radiation. In one embodiment, the mount is simple to remove because the shape of the clasp is adapted to the shape of the support. Such a mount is advantageously employed for one-piece, transparent guard devices.

In one embodiment, the mount is attached to the emitter head. In this embodiment, the guard device is a compact embodiment because of the spatial closeness of the mount to the shield and the X-ray source. The guard device rotates with the X-ray source. In this embodiment, the X-ray source or emitter head is rotatable about a subject to be examined or about an examination region without relative displacement of the guard device counter to the X-ray source. The motion of the X-ray source is not limited by mounting the guard device on the support. Increased flexibility in diagnosis is thus gained.

In one embodiment, the mount is embodied as a spring clip. The guard device includes an elastic plastic material. In alternate embodiments, additional components, for example, spring elements are attached to the shield and/or the mount. The guard device can react elastically to external stresses, such as shock or impact, which reduces the risk of injury to the patient and the risk of damage to the shield.

In one embodiment, the X-ray device is embodied as a mammography device. In this embodiment, the patient stands at the X-ray device. The patient's breast is placed on an object table and compressed by a compression unit, to keep the radiation dose as slight as possible. The other body parts of the patient can still move freely and are capable of getting into the beam region between the X-ray source and the X-ray detector. In one embodiment, when a patient is standing during the X-ray examination, guard devices are used to avoid an unwanted exposure to radiation of unexamined body parts.

### BRIEF DESCRIPTION OF THE DRAWINGS

FIG. 1 illustrates a side view according to one embodiment of an X-ray device;

FIG. 2 illustrates a front view according to one embodiment of the X-ray device of FIG. 1;

FIG. 3 illustrates a front view according to one embodiment of the X-ray device with an alternative guard device.

### DETAILED DESCRIPTION

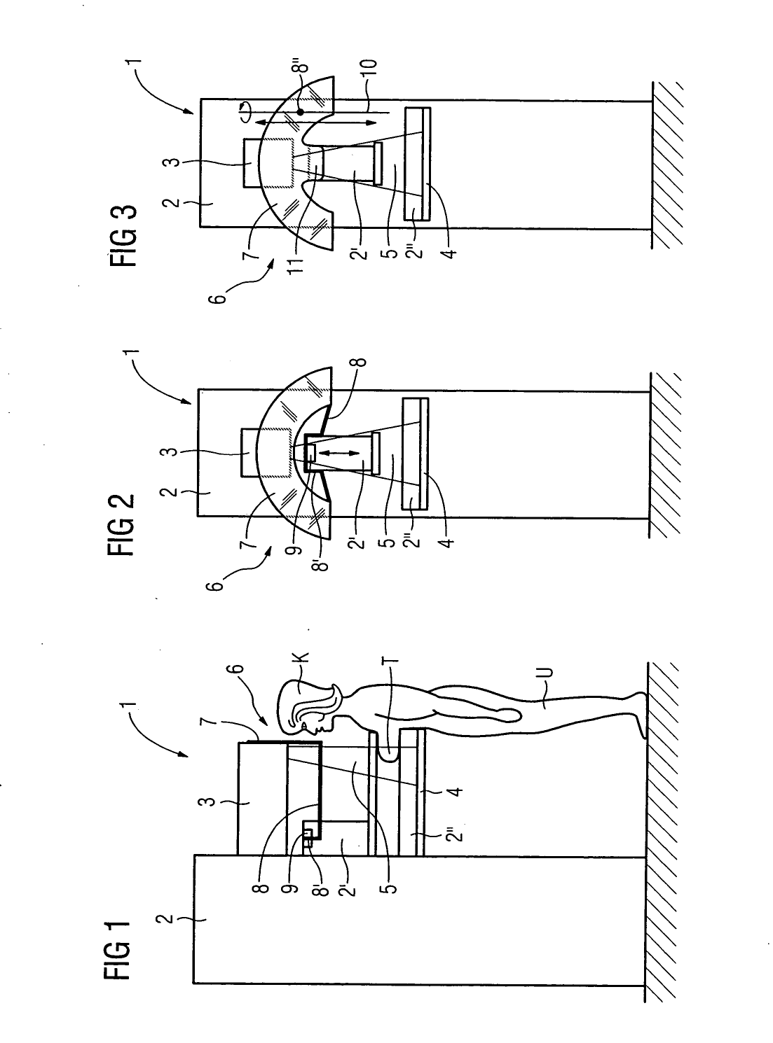

As shown in FIGS. 1 and 2, a mammography device 1 is provided for examining a subject U. -Generally, the subject U is a female patient and is positioned at the front of the mammography device 1. The subject U is omitted in FIG. 2 for the sake of better illustration of the device 1.

In one embodiment, as shown in FIG. 1, the mammography device 1 has a support 2. A compression unit 2′ and an object table 2″ are attached to the support 2. A part T to be examined is positioned on the object table 2″ and compressed by the compression unit 2′. In this embodiment, the part T is a female breast; however, the part T can be any suitable body part. The part T is located between the object table 2″ and the compression unit 2′ and between an X-ray source, in a rotatably supported emitter head 3, and an X-ray detector 4.

In one embodiment, a beam region 5 develops between the X-ray source and the X-ray detector 4 because X-radiation is emitted by the X-ray source that is integrated with the emitter head 3. In one embodiment, the beam region 5 is conical. The part T is positioned on the object table 2″ and the compression unit 2′ in such a way that the part T is located inside the beam region 5. As shown in FIG. 1, the head K of the patient U is located close to the X-ray source or to the emitter head 3.

In one embodiment, the patient's head K can enter into the beam region 5 if the patient U moves her head K forward during the examination, or in other words in the direction of the support 2, which is opposite (in front of) her. In this embodiment, a guard device 6 is provided, which comprises a shield 7 and a mount 8. The shield 7 is embodied in transparent form and has the shape of a circular-annular segment. The shield 7 can be expanded to a complete circular ring or replaced by such a ring.

In one embodiment, the material of the mount 8 and the material of the shield 7 are elastically deformable. In this embodiment, mechanical effects on the guard device 6 are dampened. The shield 7 is connected to the support 2, for example, the compression unit 2′, by a mount 8.

In one embodiment, to assist in connecting the compression unit 2′ to the shield 7, the mount 8 is adapted to the shape of the compression unit 2′ and is embodied as a clasp element 8′. In this embodiment, the clasp element 8′ is guided via the compression unit 2′ and locked by a releasable detent connection 10 when the guard device 6 is locked. This secures the position of the guard device 6.

In one embodiment, the compression unit 2′ includes an adjusting device. In this embodiment, the height of the guard device 6 can be adapted to the patient U who is to be examined. In this embodiment, if an examination is performed on the part T of the patient U, an inadvertent penetration of her head K into the beam region 5 can be prevented by the guard device 6.

In one embodiment, as shown in FIG. 3, a mammography device 1 includes a one-armed mount 8″ that is displaceable along a rail 10. In this embodiment, the mammography device 1 has a modified disposition of the guard device 6. As shown in FIG. 3, the guard device 6 includes a shield 7 and a mount 8. The mount 8 is embodied as a one-armed mount 8″ and is attached to a rail 10 attached to the support 2. The one-armed mount 8″ is displaceable along the rail 10. In this embodiment, the shield 7 can be vertically displaced to the height position of the patient U. In one embodiment, the one-armed mount 8″ of the guard device 6 is attached to the rail 10 in such a way that the one-armed mount 8″ is pivotable about the rail 10.

In one embodiment, there are two operating positions for the rotary motion. The first operating position is when the guard device 6 is located in the position that protects the patient U. The second operating position is when the longitudinal axis of the one-armed mount 8″ is aligned parallel with the front side of the support. The second operating position is achieved by a reverse pivoting motion,

In one embodiment, a large open space is quickly provided with the use of a pivotable guard device 6. For example, technicians desire the open space to access different elements of the X-ray device. In another embodiment, a spring element, not shown, may be provided on the fastening, toward the shield, of the one-armed mount 8″ so that if there is a shock to the shield 7, cushioning or dampening of the shock can be assured. The shield 7 is also supplemented with a visor 11, which further enhances the protection of the patient.

1. An X-ray device for making an image of a body part to be examined, the X-ray device comprising:
a support operative to support an emitter head having an X-ray source and an X-ray detector, wherein the body part to be examined is positioned in a beam region; and
a guard device including a shield and a mount, wherein the mount is operative to couple the shield to the support, the mount being coupled to the support at substantially the level of the shield, and wherein the guard device is operative to guard against penetration of an unexamined body part into the beam region.

2. The X-ray device as defined by claim 1, wherein the guard device comprises one piece.

3. The X-ray device as defined by claim 1, wherein at least the shield is transparent.

4. The X-ray device as defined by claim 3, wherein at least the shield is attached displaceably, rotatably, or both to the support.

5. The X-ray device as defined by claim 1, wherein the shield comprises a circular-annular segment.

6. The X-ray device as defined by claim 1, wherein the shield includes a visor.

7. The X-ray device as defined by claim 6, wherein the shield comprises a material that shields against X-rays.

8. The X-ray device as defined by claim 1, wherein a detent connection attaches the mount to the support.

9. The X-ray device as defined by claim 1, wherein a screw connection attaches the mount to the support.

10. The X-ray device as defined by claim 1, wherein a plug-in connection attaches the mount to the support.

11. The X-ray device as defined claim 1, wherein the mount comprises a clasp element, and wherein a support face of the clasp element follows the contour of the support.

12. The X-ray device as defined by claim 1, wherein the mount is attached to the emitter head.

13. The X-ray device as defined by claim 1, wherein the mount comprises a spring clip.

14. The X-ray device as defined by claim 1, wherein the X-ray device comprises a mammography device.

15. The X-ray device as defined by claim 2, wherein at least the shield is transparent.

16. The X-ray device as defined by claim 15, wherein at least the shield is attached displaceably, rotatably, or both to the support.

17. The X-ray device as defined by claim 15, wherein the shield comprises a circular-annular segment.

18. The X-ray device as defined by claim 15, wherein the shield comprises a material that shields against X-rays.

19. The X-ray device as defined by claim 15, wherein the mount is attached to the emitter head.
